(19) United States
(12) Patent Application Publication
(10) Pub. No.: US 2016/0061639 A1
(43) Pub. Date: Mar. 3, 2016

(54) Messanordnung mit einem Trägerelement und einem Sensor

(71) Applicant: ENDRESS+HAUSER FLOWTEC AG, Reinach (BL) (CH)

(72) Inventors: Achim Wiest, Weil am Rhein (DE); Christof Huber, Bern (CH); Hagen Feth, Freiburg (DE); Frank Steinhoff, Staufen im Breisgau (DE)

(21) Appl. No.: 14/652,818

(22) PCT Filed: Oct. 16, 2013
(86) PCT No.: PCT/EP2013/071617

§ 371 Date: Oct. 6, 2015

(30) Foreign Application Priority Data
Dec. 21, 2012 (DE) 102012112975.4

(51) Int. Cl. G01D 11/30; G01N 33/00

(52) U.S. Cl. CPC G01D 11/30; G01N 33/0009

(57) ABSTRACT

A measuring arrangement comprising: a support element having a longitudinal axis, wherein a sensor is arranged on the support element for ascertaining a process variable of a gaseous or liquid fluid; and the sensor, wherein the sensor has a fluid duct, and wherein the support element has a fluid duct. The characterized in that the support element has for connection of the fluid duct of the support element with the fluid duct of the sensor at least one connection element, which extends perpendicular to the longitudinal axis of the support element and into the fluid duct of the sensor.

The present invention relates to a measuring arrangement comprising a support element and a sensor as such measuring arrangement is defined in the preamble of claim 1.

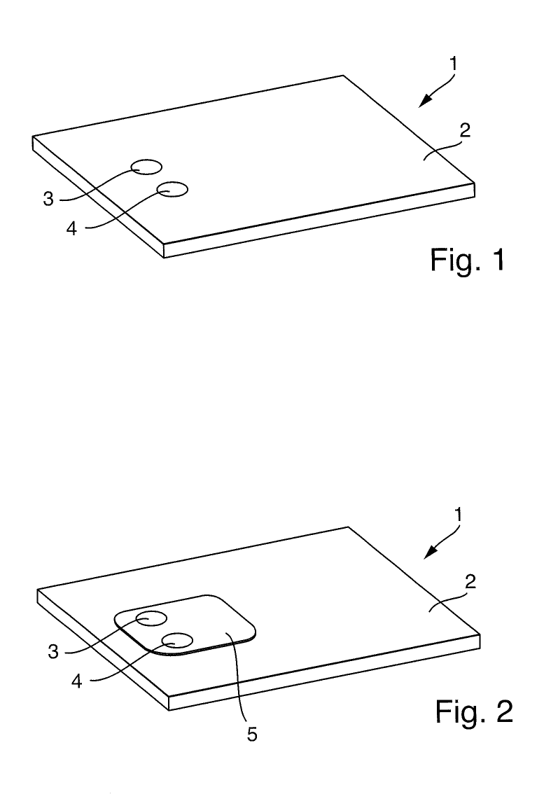

A measuring arrangement of the field of the invention composed of a support element and a micromechanical sensor is disclosed in DE 10 2011 119 472 B3. Especially in FIG. 1, this measuring arrangement has a connection between the support element and the micromechanical sensor. The connection is created by soldering. DE 10 2011 119 472 B3 additionally discloses that the solderings, referred to as chip holder, are additionally provided with a supply and/or drain line. The solderings in FIGS. 1 and 2 of this publication are, however, shown idealized. Actually, the distribution of solder and its boundary is non-uniform. The region of the fluid transfer line between micromechanical sensor and support element possesses, consequently, never the same dead volume, but, instead, can strongly vary in mass production.

It is, consequently, an object of the present invention based on DE 10 2011 119 472 B3 of the field of the invention to provide a fluid conducting connection extending between a support element and a micromechanical sensor and having a more defined dead volume.

The present invention achieves this object in the manner defined in claim 1.

A measuring arrangement of the invention includes
a) a support element having a longitudinal axis A, wherein a sensor is arranged on the support element for ascertaining a process variable of a gaseous or liquid fluid, and
b) the sensor

wherein the sensor has a fluid duct and

wherein the support element has a fluid duct, and

wherein the support element has for connection of the fluid duct of the support element with the fluid duct of the sensor at least one connection element, which extends perpendicular to the longitudinal axis A of the support element and into the fluid duct of the sensor.

The measuring arrangement of the invention provides, extending from the support element to the sensor a fluid conducting connection, which has a more defined dead volume compared with a conventional soldered connection.

Advantageous embodiments of the invention are subject matter of the dependent claims.

Advantageously, the connection element is connected as one-piece with the support element. An especially preferred one-piece connection element can be formed by means of a primary forming method, preferably by means of cold deformation, such as achieved e.g. by rolling, such as is known for vehicle construction (e.g. DE 10 2006 011 021 A1). Alternatively, the one-piece connection element can also be formed by machining.

Alternatively, the connection element can advantageously be embodied as a tubular component, which is arranged in the fluid duct of the support element. The connection element can, in this case, comprise at an exit opening of the fluid duct a metal linking layer for mechanical linking of the connection element to the support element. The metal linking layer extends over a portion of a surface of the support element. In this way, an improved pressure stability of the linking of the connection element to the support element is achieved. Additionally, the mentioned metal linking layer can extend over a portion of a surface of the sensor for mechanical linking of the connection element to the sensor. Thus, the transition between sensor, connection element and support element can be in the form of a unified linking layer.

This can be embodied as one ply or especially preferably a plurality of plies.

In the case, in which the connection element and the support element are embodied as one-piece, the metal linking layer then preferably extends only between the sensor and the connection element.

The metal linking layer, either only between sensor and connection element or between sensor, connection element and support element, can advantageously be embodied as a solder connection.

For a reliable solder connection and good reproducibility, the solder connection is formed by melting a solder wire or especially preferably a pre-blanked solder foil or an electrochemically deposited solder coating.

For a chemically-resistant solder connection, the material thereof is advantageously gold and/or tin.

Alternatively or supplementally to the solder connection, the metal linking layer can be embodied in the form of an electrochemically deposited, metal layer. Electrochemical deposition methods, especially galvanic deposition, involve only low temperatures, so that no thermal stresses arise when applying the linking layer.

There are different electrochemical deposition methods available. The electrochemically deposited, metal layer can especially advantageously be embodied as a galvanic coating. This deposition variant is especially advantageous, since the coating thickness of the linking layer can be set by adjusting the electrical current density in the galvanic bath and the deposition time.

For a targeted applying of the linking layer, it is advantageous to provide a layer of a conductive lacquer between the support element and the galvanic coating. In this case, an especially intensive deposition occurs and therewith a good anchoring of the connection elements.

The support element and/or the connection element are preferably composed of metal, preferably stainless steel, especially preferably stainless steel of type PH 17-4. This material is, on the one hand, corrosion resistant and, on the other hand, it has a thermal coefficient of expansion matched to the sensor material.

Especially advantageous is when the materials of the support element are matched in such a manner to the material of the sensor element that the thermal expansion coefficient of the material of the support element amounts to less than 7-times, preferably less than 5-times, the thermal coefficient of expansion of the material of the sensor. The same is likewise advantageous for the connection element.

For additional stabilizing it is advantageous when between the sensor and the support element other material bonded connections are arranged. These material bonded connections can especially be solder connections.

Especially advantageous is when the aforementioned material bonded connections are distributed as uniformly as possible in the region between the sensor and the support element. Therefore, it is advantageous when the surface of the sensor facing the support element is divided into at least three equally dimensioned sensor sections, wherein at least two of the three sensor sections each has at least one of the material bonded connections.

The invention will now be explained in greater detail based on the appended drawing, the figures of which show as follows:

FIG. 1 a schematic representation of a sensor with sensor body and connection openings,

FIG. 2 a schematic representation of the sensor body with a first galvanic layer on a part of the surface of the sensor body for electrically connecting the connection openings,

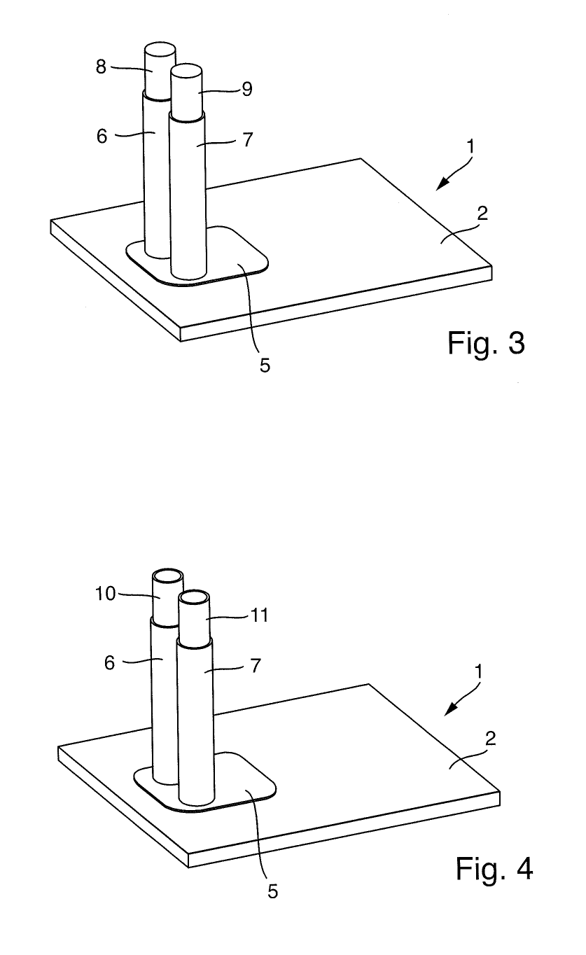

FIG. 3 a schematic representation of the sensor body with conduits and stoppers arranged on the connection openings,

FIG. 4 a schematic representation of the sensor body with conduits and tubes arranged on the connection openings,

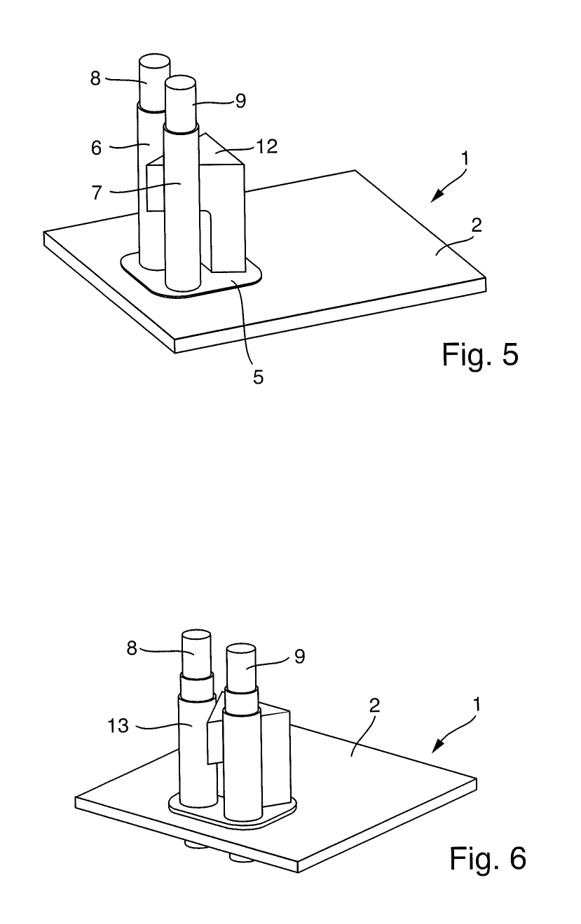

FIG. 5 a schematic representation of the sensor body with conduits, which are supported by means of a support against a first lateral surface of the sensor body,

FIG. 6 a schematic representation of the sensor body with galvanic coatings on a surface of the sensor body and a lateral surface of each conduit,

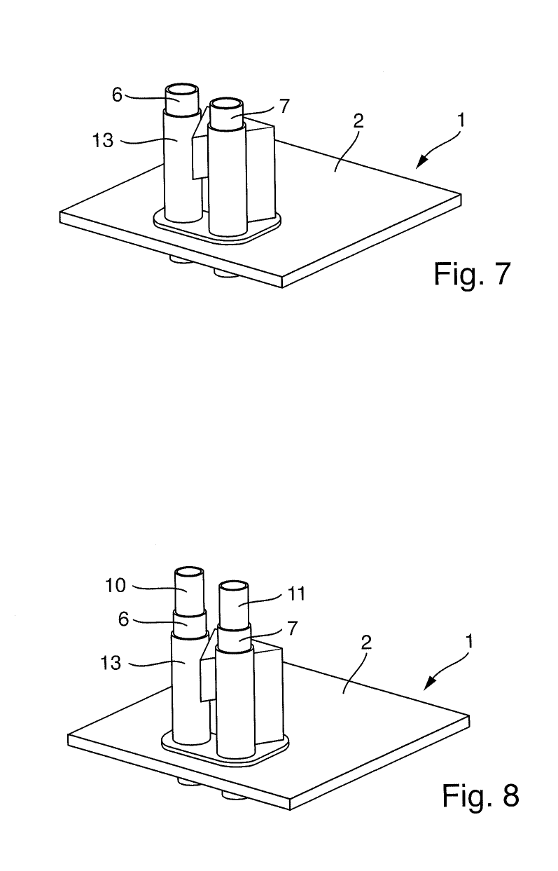

FIG. 7 a schematic representation of the sensor body with conduits without stopper, with galvanic coatings on the surface of the sensor body and the lateral surface of each conduit, and

FIG. 8 a schematic representation of the sensor body with conduits and tubes, with galvanic coatings on the surface of the sensor body and the lateral surface of each of the conduits.

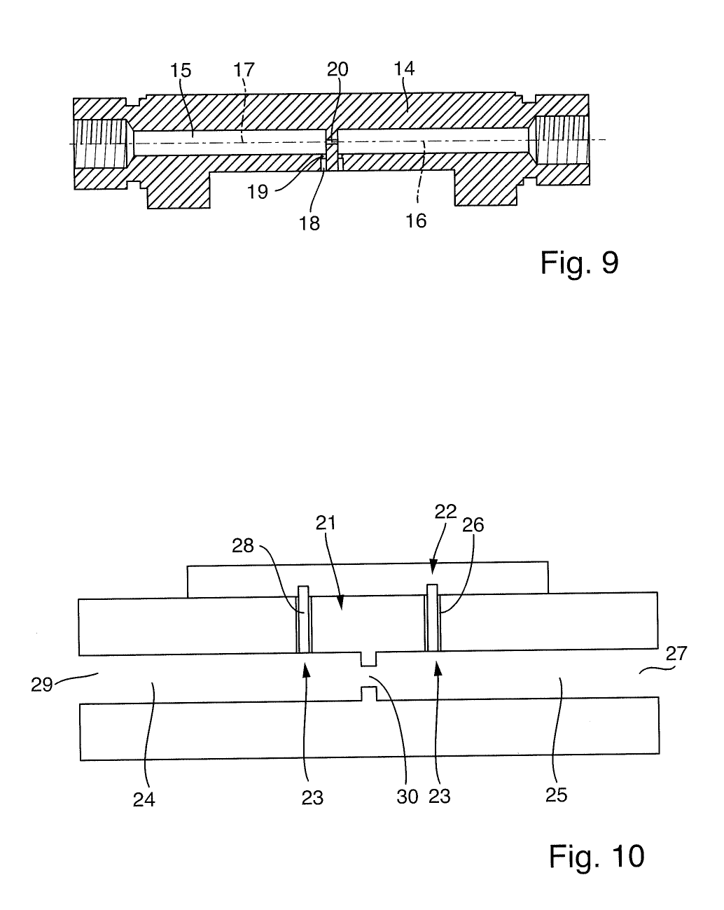

FIG. 9 a sectional view of a support element of a first measuring arrangement;

FIG. 10 schematic sectional view of the measuring arrangement;

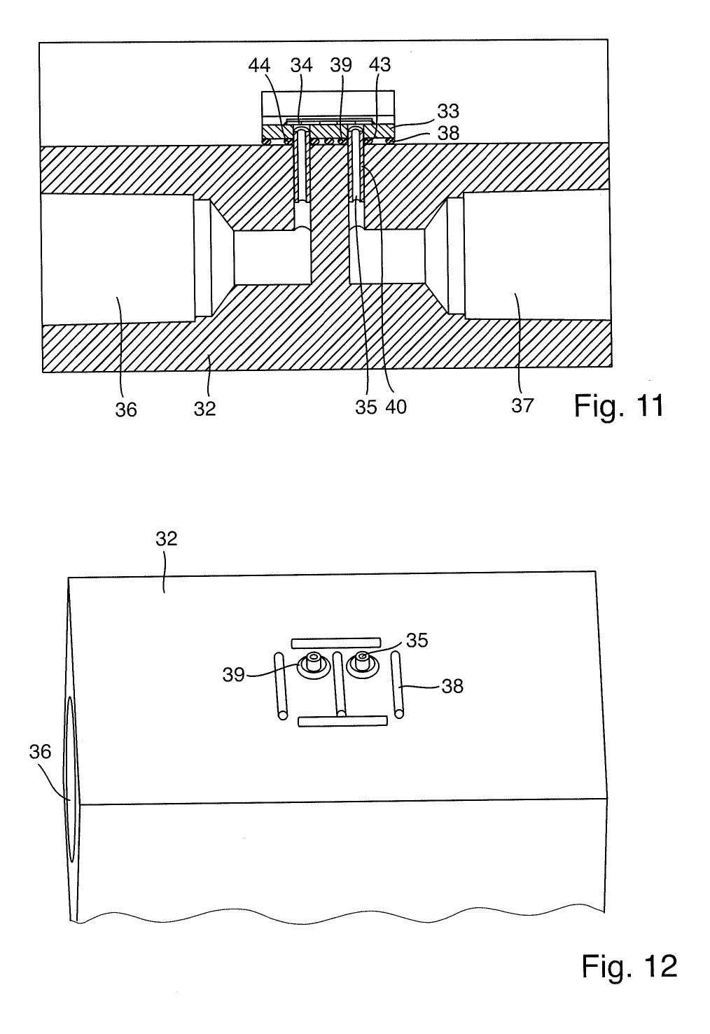

FIG. 11 schematic sectional view of the measuring arrangement in perspective;

FIG. 12 schematic representation of the support element of the measuring arrangement with a metal linking layer; and

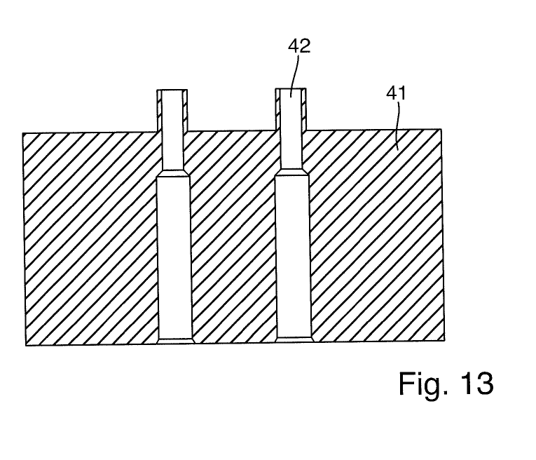

FIG. 13 schematic representation of a second measuring arrangement.

The measuring arrangements illustrated in FIGS. 1-13 are preferably applied in measuring devices of process and automation technology.

The present invention relates to linking a sensor to a support element. The sensors in the following examples of embodiments are micromechanical sensors. The invention, is however, not limited to micromechanical sensors.

The base surface of a preferred sensor can be preferably the maximum base surface of a wafer. The base surface is the surface, with which the sensor can be connected with the support element.

Especially preferably, at least one edge length of the sensor is less than or equal to 10 cm. Quite especially preferably, all edge lengths of the sensor are less than or equal to 10 cm.

FIG. 1 shows a second example of an embodiment of a sensor 1 intended for a Coriolis mass flow measuring device and embodied in micromechanical construction (MEMS—micro electro mechanical system). Sensor 1 includes a sensor body 2, which is embodied of ceramic and has a surface with a first connector opening 3 and a second connector opening 4, each of which has a diameter of about 1 mm. Sensor body 2 is prismatic and includes first and second, rectangular, lateral surfaces, each of which is typically about 1 cm² in area. The first and second connector openings 3, 4 are arranged on a first lateral surface of the sensor body 2 and lead to a flow accepting volume arranged in the interior of the sensor body and limited from the sensor body by means of a metal body, especially a metal tube.

FIG. 2 shows a first method step of the invention. In this first method step, a part of the first lateral surface of the sensor body 2 is galvanized in such a way that the galvanic layer 5 establishes an electrical connection both to the first as well as also to the second connector opening 3, 4. Alternatively, the entire surface of the sensor body 2 can be galvanized. In this way, the electrical and electronic components of the sensor 1 are protected against electromagnetic fields reigning outside of the sensor body 2.

FIG. 3 shows the next method step of the invention. First and second cylindrical conduits 6, 7, each of which has a nominal diameter of typically about 1 mm and each of which has a stopper, respectively first and second stoppers 8, 9, are positioned, respectively, perpendicularly on the first and second connector openings 3, 4. The first and second stoppers 8, 9 are cylindrically embodied and have an outer diameter, which corresponds to an inner diameter of the conduits 6, 7 and extend, in each case, partly out of the first and second conduits 6, 7, wherein the extending out parts of the first and second stoppers 8, 9 are at ends of the first and second conduits 6, 7 lying opposite the first and second connector openings 3, 4.

The first and second stoppers 8, 9 serve for closing the first and second conduits 6, 7 during galvanizing, so that no electrolytic liquid can get into the first and/or second conduits 6, 7. Alternatively, the first and second stoppers 8, 9 can also be arranged directly on the first and second connector openings 3, 4.

FIG. 4 shows, that instead of the first and second stoppers 8, 9, internal first and second tubes 10, 11 can be used.

FIG. 5 shows that after positioning the first and second conduits 6, 7, these are supported by means of a support 12 against the first lateral surface of the sensor body 2. Support 12 can be arranged e.g. by means of a second galvanic layer on the lateral surface of the sensor body.

FIG. 6 shows an additional method step of the invention, in the case of which a sealing galvanic coating 13 is produced on the galvanic layer 5 and on lateral surfaces of the first and second conduits 6, 7. Since the first lateral surface of the sensor body 2 is composed of ceramic and only partially has a galvanic layer 5, the galvanizing of the first and second connector openings 3, 4 covers only the part of the first lateral surface of the sensor body 2, which has a galvanic layer. The galvanizing produces an electrical connection between the first and second conduits 6, 7 and the first and second connector openings 3, 4. In this way, electrical fields produced by friction of the flowing medium and arising in the interior of the sensor body 2, can drain via the first and second conduits 6, 7.

If the connection openings 3, 4 are sealed by means of the stoppers 8, 9, the galvanic coating 13 is produced on the galvanic layer 5 and the lateral surfaces of the first and second stoppers 8, 9. After removal of the first and second stoppers 8, 9, the coating 13 forms a communicating connection to the first and second connector openings and, thus, to the inner volume of the sensor body 2.

FIG. 7 shows an additional method step of the invention: After producing a sealing galvanic coating 13 between a part of the surface of the sensor body 2 and the first and second conduits 6, 7, the first and the second stoppers 8, 9 can be removed. This can happen mechanically or by a dissolving and/or melting process.

If instead of the first and second stoppers 8, 9, first and second tubes 10, 11 are used, then these are not removed (see FIG. 8). The first and second tubes 10, 11 can then be connected to extending hoses.

The method of the invention creates a communicating connection between the interior flow accepting volume of the sensor body 2 and the first and second conduits 6, 7, so that a pressure change of a medium in the first conduit 6 is transmitted via the interior volume of the sensor body 2 into the second conduit 7.

Then, the sensor described in FIGS. 1-8 can be connected with a support element to form a measuring arrangement of the invention.

The conduits 6, 7 can, in such case, also be connection elements, which are secured to a support element illustrated in FIG. 9 before the securement in the sensor body. The mounting of the conduits, respectively connection elements, onto the support element will now be explained in greater detail.

FIG. 9 shows a corresponding support element 14 having a longitudinal axis A. A sensor for ascertaining a process variable of a gaseous or liquid fluid can be arranged on support element 14. Support element 14 includes a fluid duct, which in the present example is divided into a fluid supply duct 15 and a fluid drain duct 16 for supplying and removing a fluid to and from the sensor. There are, however, also other support element, sensor designs possible, for example, a pressure sensor, in the case of which the fluid supply and drain can be combined into one duct.

The process variables can preferably include density, viscosity, substance composition, temperature, pH-value, conductivity, particle content, volume flow, mass flow and/or flow velocity of a fluid.

The fluid supply duct includes in the example of an embodiment illustrated in FIG. 9 a first duct segment 17, which extends essentially parallel to the longitudinal axis A of the support element 14. This duct segment is terminally connectable with a process connection of a pipeline. The fluid supply duct additionally includes a second duct segment 18 and the first duct segment 17 is in communication with the second duct segment 18. The second duct segment 18 in the present example of an embodiment is arranged at an angle of 90° to the longitudinal axis of the support element 14. In such case, the diameter of the first duct segment 17 is greater, preferably at least two times greater, than the diameter of the second duct segment 18. The second duct segment 18 includes a diameter widening 19 for accommodating a connection element. After insertion of the connection element, there is no nominal diameter jump within the second duct segment 18. The fluid can be drained radially to the axis out of the support element through the second duct segment 18.

Support element 14 includes as part of the fluid duct additionally the fluid drain duct 16, which is essentially of equal construction to the fluid supply duct 15. Between the fluid drain duct and the fluid supply duct, optionally a duct connection segment 20 can be arranged, which is arranged in the support element 14 parallel to the longitudinal axis A and connects the fluid supply duct and the fluid drain duct with one another. Thus, not the entire fluid stream needs to be led through the sensor, but, instead, only a part of the fluid. The nominal diameter of the duct connection segment is, in such case, smaller, preferably at least two times smaller, than the nominal diameter of the first duct segment 17.

FIG. 10 shows a schematic construction of a first measuring arrangement 31. A measuring arrangement of the invention includes at least one support element 21, a micro electromechanical sensor 22 and connection elements 23, which connect the support element 21 and the sensor 22 with one another, in such a manner that the connection elements 23 protrude inwardly into the micro electromechanical sensor 22, respective into a fluid duct arranged therein.

Micro electromechanical sensors, such as can be applied in the present example, are known per se. A sensor installed in the present example can be a Coriolis flow measuring device, a magneto-inductive flow measuring device, a thermal, flow measuring device, a pressure measuring device, a viscosity measuring device, a spectroscopic measuring device, an ultrasonic measuring device, especially an ultrasonic, flow measuring device, a density measuring device and ascertain process variables such as viscosity, density, pressure, substance composition, temperature, viscosity, pH-value, conductivity, particle content and/or, in given cases, also flow. A sensor in the context of present invention can also be a chromatographic analyzer (an LC- or a GC analyzer). These can likewise be implemented in micro electromechanical construction.

The support element 21 includes a fluid supply duct 24 and a fluid drain duct 25. These have, in each case, a first duct segment 27, 29 parallel to the longitudinal axis A and a second duct segment 26 and 28, which extends radially to the longitudinal axis through the support element 21. Also present in the example of an embodiment shown in FIG. 10 is a duct connection segment 30 extending between the respective first duct segments 27 and 29 of the fluid supply duct 24 and the fluid drain duct 25.

In contrast to FIG. 9, support element 21 has no diameter widening 19. Rather, the connection elements 23 are inserted completely into the second duct segments 26 and 28.

The micro electromechanical sensor is preferably manufactured of a glass or silicon material. Typically, the coefficient of thermal expansion in the case of these materials amounts to, for instance, 3*10⁻⁶ K⁻¹. Alternatively, also sensors of ceramic materials are applicable for such use. The connection elements 23 are either embodied as specialized components in the form of small tubes or integrally formed, such as explained in greater detail with reference to FIG. 13. They are preferably manufactured of stainless steel—preferably type PH 17-4. Other materials, for example, synthetic materials, e.g. plastics, provide other options. Especially in the case of hot or cold fluids, it is, however, advantageous, when the thermal expansion coefficients of the materials of the sensor and of the connection element differ by no more than 5-times from one another. Otherwise, unsealed locations can occur at higher pressures or even the sensor can disconnect. Stainless steel of type PH 17-4 fulfills these requirements relative to a silicon material and/or glass material (including borosilicate). To the extent that the connection elements are embodied integrally with the support element, the material of the support element should naturally correspond to the material of the connection elements. To the extent that, however, the connection elements 23 are provided in the support element 21 as separate components, the material of the support element can preferably be selected in the form of a more cost effective material, for example, stainless steel of type 316 L. Alternatively, also other materials, especially titanium, aluminum, zirconium, tantalum, silicon or conducting ceramic material can be applied for the support element and/or the connection element.

Additionally to the metal linking layer, also a plastics layer can be provided, which protects the linking layer against oxidative attack. In such case, it can preferably be a copolymer.

In a special embodiment, an internal coating of the connection element or the connection element, as a whole, is composed of a synthetic material selected from the following materials: PE, PEEK, PFA, PTFE, PBT PEK. In this case, however, in order to provide a galvanic coating, first of all, an electrically conductive layer must be applied by sputtering, metallizing or vapor deposition.

Additionally or alternatively, also heat conductive substances, which increase the thermal conductivity of the metal linking layer, can be included in the linking layer, in order to enable a thermal contacting between the support element and the sensor.

Additionally or alternatively, also magnetic substances can be included in the synthetic material, in order to enable magnetic contacting between sensor and support element. Corresponding magnetic substances can be e.g. particles of magnetite.

Also, metal elements, for example, conductive traces, which improve the electrical conductivity, can be contained in the metal linking layer.

Additionally advantageously arranged between the support element and the connection element and the support element and the sensor can be a seal in the form of a membrane structure or a sealing lip, so that the solder connection is not mechanically or chemically excessively loaded.

To the extent that an aforementioned solder connection is created, it is advantageous to pretreat the surfaces to be connected, in order to enable better adhesion. This can occur chemically by etching or through corona treatment or lasers or by abrasive methods such as e.g. sand blasting. The treated surfaces can then be wetted better by the solder.

The connection elements 23 enable especially a flow connection between micro electromechanical sensor 22 and the support element 21. However, it is advantageous, especially in the case of higher pressures, to provide an additional mechanical linking of the micro electromechanical sensor 22.

An embodiment of the mechanical linking will now be explained in greater detail with reference to FIGS. 11 and 12. FIGS. 11 and 12 show analogously to FIG. 10 a support element 32 with a fluid supply duct 36 and a fluid drain duct 37. Arranged in these ducts 36 and 37 are respective connection elements 35. FIG. 11 shows additionally a micro electromechanical sensor 33 and, arranged therein, a fluid duct 34, which extends from a fluid inlet 44 to a fluid outlet 43, into which the connection elements 35 protrude.

The mechanical linking of the micro electromechanical sensor occurs in the example of an embodiment of FIGS. 11 and 12 by means of a solder connection. This solder connection is applied in FIGS. 11 and 12 in the form of solder wires 38 and solder rings 39 on the support element 32. The solder rings 39 achieve a mechanical and at the same time pressure stable and medium excluding connection of the connection elements 35 with the support element 32.

The linking between the micro electromechanical sensor and the support element can alternatively to a solder connection also occur by an adhesive system, e.g. by means of an epoxy resin. The solder connection is, however, especially resistant to acids and alkaline solutions.

Besides the solder rings 39, also solder wires are applied on the support element 32, in order to enable a direct connection with the micro electromechanical sensor 33.

Suited as solder material is especially preferably a noble metal, e.g. silver or gold or alloys thereof. Also, for example, eutectic mixtures of silver and tin can be used. The shrinkage of these materials amounts, in such case, to preferably less than 1 vol %.

Alternatively or supplementally to the solder rings and solder wires, also blanked metal foils, especially gold and/or tin foils, and/or an electrochemically deposited layer or layers, especially a gold layer, can be used for obtaining a secure linking. The solder can additionally be applied by means of a template on the support material.

The electrochemical deposition can be masked for a more targeted application of the layer on a part of the surface of the support element 32. This assures a defined height of the solder and a defined volume of the solder.

Alternatively, however, less preferably than gold material, also tin material or alloys of both materials can be utilized for forming the solder connections. Both gold as well as also tin have a good chemical resistance to most fluids. The shrinkage of these materials amounts, in such case, to preferably less than 1 vol %.

In such case, it is advantageous, when the solder layer is less than ⅕ mm, preferably less than 1/10 mm.

An electrochemical deposition of a metal layer can analogously to the example of an embodiment described with reference to FIGS. 1-8 occur by means of a galvanic deposition.

Alternatively, a multilayer electrochemical deposition can occur, wherein the gold layer or tin layer is only the uppermost layer toward the sensor.

In the case of a galvanic deposition of a metal linking layer on the sensor, a conductive lacquer, preferably a silver or graphite conductive lacquer, can be applied on the support element and/or the connection elements for improving deposition rate and adhesion.

Also a connection between the micro electromechanical sensor and one of the connection elements 35 can be achieved analogously to the connection between the support element 32 and one of the connection elements 35.

Especially because of its mechanical stability, in such case, a unified metal linking layer is applied, which extends from the support element 32 over the connection element 35 and to the micro electromechanical sensor 33.

The fluid conducting connection shown in FIGS. 1-13 provided by the connection element has preferably a cross sectional area of less than 100 mm² and especially less than 20 mm².

A preferred coating thickness of the linking layer amounts to less than 1 mm, preferably less than 200 μm and especially preferably less than 100 μm. An especially preferred coating thickness of the mechanical linking layer lies in the range between 100 nm and 100 μm,

FIG. 13 shows for linking the sensor element to the support element 41 connection elements 42, which are, however, integrally connected with the support element 41.

This integral construction is especially advantageous, since such an embodiment directly prevents an unsealed location. A possible manufacturing of such an integrally formed connection element can involve, for example, a cold forming method, especially milling.

By arranging connection elements between the support element and the micro electromechanical sensor and the additional plugging into the micro electromechanical sensor, a more defined dead volume is created compared with a conventional solder stop. Additionally, a more reproducible and chemically more resistant connection is achieved, which is additionally more resistant to shear forces.

In such case, the connection element in the case of application of a solder acts as a defined solder stop without a dead volume forming.

The thus created linking of a sensor, for example, a sensor of micro electromechanical construction, to the support element is preferably pressure stable up to a pressure of greater than 20 bar, preferably greater than 80 bar.

Substances, which improve the electrical, thermal and/or magnetic conductivity of the linking layer, can be added to the metal linking layer. Alternatively or supplementally, also substances, which enable a better thermal expansion accommodation between the materials of the support element and of the sensor, can be added to the metal of the linking layer. Substances for improving the electrical conductivity are preferably solderable and at the same time conductive, for instance the already earlier mentioned substances.

Substances, which can improve the thermal conductivity are, for example, silicon carbide and/or aluminum nitride.

Substances, which enable a better thermal expansion accommodation, can be preferably corundum and/or aluminum oxide.

Substances, which can improve the magnetic conductivity, are, for example, magnetite or magnetizable metals or metal alloys.

### LIST OF REFERENCE CHARACTERS

1 sensor

2 sensor body

3 first connector opening

4 second connector opening

5 first galvanic layer

6 first conduit

7 second conduit

8 first stopper

9 second stopper

10 first tube

11 second tube

12 support

13 galvanic coating

14 support element

15 fluid supply duct

16 fluid drain duct

17 first duct segment

18 second duct segment

19 diameter expansion

20 duct connection segment

21 support element

22 micro electromechanical sensor

23 connection element

24 fluid supply duct

25 fluid drain duct

26 second duct segment

27 first duct segment

28 second duct segment

29 first duct segment

30 duct connection segment

31 measuring arrangement

32 support element

33 micro electromechanical sensor

34 fluid duct

35 connection element

36 fluid supply duct

37 fluid drain duct

38 solder wires

39 solder rings

40 fluid outlet

41 support element

42 connection element

43 fluid outlet

44 fluid inlet

1-15. (canceled)

16. A measuring arrangement, comprising:
a support element having a longitudinal axis; and
a sensor arranged on said support element for ascertaining a process variable of a gaseous or liquid fluid, wherein:
said sensor has a fluid duct, which extends within said sensor;
said support element has a fluid duct, said support element has for connection of said fluid duct of said support element with said fluid duct of said sensor at least one connection element, which extends perpendicular to the longitudinal axis of said support element and into said fluid duct of said sensor.

17. The measuring arrangement as claimed in claim 16, wherein:
said connection element is connected as one-piece with said support element.

18. The measuring arrangement as claimed in claim 16, wherein:
said connection element is formed as part of said support element.

19. The measuring arrangement as claimed in claim 16, wherein:
said connection element is embodied as a tubular component, which is arranged in the fluid duct of said support element.

20. The measuring arrangement as claimed in claim 19, wherein:
said connection element has at an exit opening of said fluid duct for mechanical linking of said connection element to said support element a metal linking layer, which extends over a portion of a surface of said support element.

21. The measuring arrangement as claimed in claim 17, wherein:
a metal linking layer or the metal linking layer as claimed in claim 20 extends over a portion the surface of said sensor for mechanical linking of said connection element to said sensor.

22. The measuring arrangement as claimed in claim 21, wherein:
said metal linking layer is embodied as a solder connection.

23. The measuring arrangement as claimed in claim 22, wherein:
said solder connection is produced by melting a solder wire or especially preferably a pre-blanked solder foil or an electrochemically deposited solder coating.

24. The measuring arrangement as claimed in claim 23, wherein:
at least one material component of said solder connection is a noble metal, especially gold, and/or tin.

25. The measuring arrangement as claimed in claim 20, wherein:
said metal linking layer is embodied in the form of an electrochemically deposited, metal layer.

26. The measuring arrangement as claimed in claim 23, wherein:
said electrochemically deposited, metal layer is embodied as a galvanic coating and that a layer of a conductive lacquer is arranged between said support element and said galvanic coating.

27. The measuring arrangement as claimed in claim 16 wherein:
said support element and/or said connection element is composed of metal, preferably stainless steel, especially preferably stainless steel of type PH 17-4.

28. The measuring arrangement as claimed in claim 16, wherein:
the thermal expansion coefficient of the material of said support element amounts to less than 5-times, preferably less than 4-times, the thermal coefficient of expansion of the material of said sensor (1, 22, 33).

29. The measuring arrangement as claimed in claim 16, wherein:
between said sensor and said support element other material bonded connections are arranged, wherein the material bonded connections are especially embodied as solder connections.

30. The measuring arrangement as claimed in claim 28, wherein:
the surface of said sensor facing said support element is divided into at least three equally dimensioned, sensor sections, wherein at least two of the three sensor sections each has at least one of the respective material bonded connections.
